(19) United States
(12) Patent Application Publication
(10) Pub. No.: US 2009/0143484 A1
(43) Pub. Date: Jun. 4, 2009

(54) USE OF GARLIC OIL TO INCREASE BIOAVAILABILITY OF COENZYME Q-10

(76) Inventors: Jingang Shi, Beijing (CN); Yunlong Feng, Tianjin (CN); Chenghai Zhao, Beijing (CN); Wenxian Pan, Beijing (CN)

Correspondence Address: FULBRIGHT & JAWORSKI L.L.P.;Attn: MN IP Docket, 600 Congress Avenue, Suite 2400, Austin, TX, 78701, US

(21) Appl. No.: 12/325,380

(22) Filed: Dec. 1, 2008

Related U.S. Application Data
(60) Provisional application No. 60/991,972, filed on Dec. 3, 2007.

(51) Int. Cl. A61K 31/045

(52) U.S. Cl. 514/729

(57) ABSTRACT

The invention describes compositions that include a sulfide containing molecule, such a garlic oil, and a coenzyme Q molecule. The sulfide containing molecule solvates the coenzyme Q molecule, thus enhancing the bioavailability of the coenzyme Q molecule in a subject in need thereof, relative to administration of coenzyme Q devoid of the presence of a sulfide containing molecule.

### CROSS REFERENCE TO RELATED APPLICATIONS

This application claims priority to U.S. Provisional Patent Application No. 60/991,972, filed Dec. 3, 2007, entitled “Use of Garlic Oil to Increase Bioavailability of Coenzyme Q-10”, the entire content of which is hereby incorporated by reference in its entirety.

### FIELD OF THE INVENTION

The invention relates generally to unique combinations of garlic oil or one or more of its isolated or purified components and a coenzyme Q.

### BACKGROUND OF THE INVENTION

CoQ-10 (coenzyme Q10) is a fat-soluble quinone, a benzoquinone that is structurally similar to vitamin K and commonly known as ubiquinone. CoQ-10 is found in most living organisms, and is essential for the production of cellular energy. CoQ-10 (2,3 dimethyl-5 methyl-6-decaprenyl benzoquinone) (2-(all-E)-3,7,11,15,19,23,27,31,35,39-Decamethyl-2,6,10,14,18,22,26,30,34,38-tetracontadecaenyl)-5,6-dimethoxy-3-methyl-p-benzoquinone) is an endogenous antioxidant found in small amounts in meats and seafood. Although CoQ-10 is found in all human cells, the highest concentrations of CoQ-10 occur in the heart, liver, kidneys, and pancreas. It is found naturally in the organs of many mammalian species.

CoQ-10 is an important nutrient because it lies within the membrane of a cell organelle called the mitochondria. Mitochondria are known as the “power house” of the cell because of their ability to produce cellular energy, or ATP, by shuttling protons derived from nutrient breakdown through the process of aerobic (oxygen) metabolism. CoQ-10 also has a secondary role as an antioxidant. CoQ-10, due to the involvement in ATP synthesis, affects the function of almost all cells in the body, making it essential for the health of all human tissues and organs. CoQ-10 particularly effects the cells that are the most metabolically active: heart, immune system, gingiva, and gastric mucosa

CoQ-10 is sparingly soluble in most hydrophilic solvents such as water. Therefore, CoQ-10 is often administered in a powdered form, as in a tablet or as a suspension. However, delivery of CoQ-10 by these methods limits the bioavailability of the material to the individual.

Several clinical trials have shown CoQ-10 to be effective in supporting blood pressure and cholesterol levels. Furthermore, CoQ-10 has also been shown to improve cardiovascular health. CoQ-10 has been implicated as being an essential component in thwarting various diseases such as certain types of cancers. These facts lead many to believe that CoQ-10 supplementation is vital to an individual's well being.

Reduced benzoquinones are known to be effective reductants for oxygen or lipid radicals. Some studies have shown that reduced CoQ-10 (ubiquinol) is an effective antioxidant. In fact, reduced CoQ-10 now appears to function as part of a complex chain of antioxidant activity. Apparently, reduced CoQ-10 plays a role in the reduction of radicals of alpha-tocopherol and ascorbate formed when these antioxidants are oxidized by oxygen or carboxyl radicals present in physiological systems. There are no known enzymes for direct reduction of a tocopheryl radical or an external ascorbate radical, but there are enzymes in all membranes that can reduce CoQ-10 and thus reduced CoQ-10 can subsequently reduce the tocopheryl or ascorbate radicals to provide tocopherol or ascorbate. Without the support of enzymes to reduce CoQ-10, the reduced CoQ-10 would not be a very effective antioxidant as the semiquinone formed by interaction with lipid or oxygen radicals is readily autooxidized with formation of a superoxide radical.

Therefore, a need exists for methods and compositions that provide CoQ-10 or reduced CoQ-10 in a form that can be assimilated and retains antioxidant activity.

### BRIEF SUMMARY OF THE INVENTION

The present invention surprisingly provides compositions that include a coenzyme Q, a reduced coenzyme Q or mixtures thereof, and a sufficient amount of a sulfide containing material suitable to dissolve the coenzyme Q, reduced coenzyme Q or mixtures thereof. Dissolution of the coenzyme Q material in the sulfide containing material provides increased bioavailability of the coenzyme Q material relative to an equivalent amount of the coenzyme Q material without the sulfide containing material. In general, the bioavailability of the coenzyme Q material is increased from about 15 percent to about 1500 percent versus equivalent coenzyme Q formulations without the sulfide containing material.

In certain aspects of the invention, garlic oil is used as the sulfide containing material.

In certain aspects of the invention, the combination of garlic oil (or one or more of the individual sulfide containing components) and a coenzyme Q material helps reduce the oxidation of LDL. Therefore, in one aspect, the combination of garlic oil (or a sulfide containing component thereof) and a coenzyme Q material can be used to treat, reduce or prevent oxidation of LDL. Administration of an effective amount of the combination helps to inhibit the oxidation of LDL in a physiological environment. Consequently, the combination of the coenzyme Q material and a sulfide containing material(s), such as garlic oil, can be used to treat or prevent various diseases such as diabetes, atherosclerosis and or cardiovascular diseases.

In other aspect of the invention, components of garlic oil are used as sulfide containing materials, suitable to dissolve the coenzyme Q material. These components of garlic oil suitable to dissolve the coenzyme Q material include, for example, diallylsulfide and/or diallyldisulfide.

In another aspect, the present invention provides a soft gelatin capsule that encapsulates the coenzyme Q/sulfide containing material compositions described herein.

The compositions and soft gelatin capsules can further include various carriers and additives, such as suitable antioxidants and/or vitamins.

The present invention also provides a method to prepare solutions of a coenzyme Q material and a sulfide containing material suitable to dissolve the coenzyme Q material.

The present invention further provides methods to treat various conditions associated with decreased levels of a coenzyme Q (e.g., coenzyme Q-10), such as mitochondrial related diseases and disorders, Parkinson's disease, Prater-Willey syndrome, cardiovascular disease, congestive heart failure, migraine headaches or headaches by administering to the individual in need thereof, an effective amount of any of the compositions disclosed herein.

In still another aspect, the present invention also provides packaged nutraceuticals that are disclosed herein.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 provides the %-uptake of CoQ10 from various sample preparations.

FIG. 2 provides maximum absorption of CoQ10 from various sample preparations.

FIG. 3 provides %-uptake of CoQ10 from reduced sample preparations.

FIG. 4 demonstrates that reduction of oxidation of LDL by garlic oil.

FIG. 5 demonstrates the reduction of oxidation of LDL by coenzyme Q-10.

FIG. 6 demonstrates the synergistic effect of the combination of garlic oil and coenzyme Q-0 on LDL.

FIG. 7 demonstrates the relative equivalency of coenzyme Q-10/garlic oil and the combination of both on the oxidation of LDL.

### DETAILED DESCRIPTION

The present invention surprisingly provides the ability to solvate coenzyme Q materials with a sufficient amount of one or more sulfide containing materials. Use of sulfide containing materials provide increased bioavailability of the coenzyme Q material relative to an equivalent amount of the coenzyme Q material that is presented without one or more sulfide containing materials.

The percentage of the increased bioavailability of the coenzyme Q material treated with one or more sulfide containing material(s) with respect to an equivalent amount of a coenzyme Q material without one or more sulfide containing materials ranges from about 15 percent to about 1500 percent, in particular from about 25 percent to about 500 percent, more particularly from about 50 to 250 percent and even more particularly from about 100 to about 200 percent. It should be understood that these ranges are inclusive and include various ranges that fall from 15 percent and 1500 percent, such as for example, 16 percent to about 199 percent, 17 percent to about 143 percent, 23 percent to about 187 percent etc.

In the specification and in the claims, the terms “including” and “comprising” are open-ended terms and should be interpreted to mean “including, but not limited to . . . ” These terms encompass the more restrictive terms “consisting essentially of” and “consisting of:”

The term “coenzyme Q material” is intended to include both coenzyme Q compounds (as an oxidized form known as a ubiquinone) and reduced coenzyme Q compounds (reduced forms known as ubiquinols) as well as mixtures thereof.

The term “coenzyme Q” or “ubiquinone” (CoQ-10) is used throughout the present specification to describe a group of lipid soluble benzoquinones involved in electron transport in mitochondrial preparations, i.e., in the oxidation of succinate or reduced nicotine adenine dinucleotide (NADH) via the cytochrome system. The compounds can be described as: coenzyme Qₙ where n is 1-12 or ubiquinone (x) in which x designates the total number of carbon atoms in the side chain and can be any multiple of 5. Differences in properties are due to the difference in the chain length. In particular, ubiquinone for use in the present invention is the reduced form of coenzyme Q10, known as ubiquinol. Therefore, the term CoQ-10 includes all variations where n is from 1 to 12. Likewise, reduced CoQ-10 also includes all variation where n is from 1 to 12.

The term “ubiquinol” is used throughout the specification to describe the reduced form of coenzyme Qₙ that is used as the active agent in compositions according to the present invention. In ubiquinol, the quinone ring of coenzyme Qₙ is reduced such that the structure of the compound appears as set forth below. In one aspect, ubiquinol, n is preferably 10 and is derived from coenzyme Q₁₀. The amount of ubiquinol which is included in compositions according to the present invention ranges from about 0.1% to about 50% by weight of the final composition which is encapsulated in a soft gelatin capsule, more preferably about 0.5% to about 10% by weight, even more preferably about 1% to about 5% by weight. The amount of ubiquinol which is included in compositions to be encapsulated ranges from about 0.1 to about 10.0 times, more preferably about 1 to about 3 times the amount (in weight percent) of the lipid soluble reducing agent which is included in compositions according to the present invention.

It should be understood, that throughout this specification, reference to CoQ-10 and reduced CoQ-10 refers to all possible derivatives where n is as detailed above.

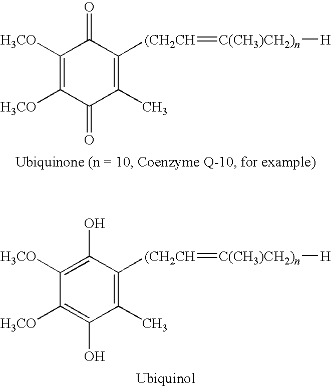

Dihydrolipoic acid (DHLA) is a constituent of cellular metabolism and can be used as a solvent for coenzyme Q materials as well as an antioxidant. DHLA has two thiol residues that make is susceptible to radical species, thus provides antioxidant functionality to the biomolecule. Oxidation reduction (redox reactions) involves the transfer of an electron from a donor to an acceptor. When the donor loses an electron, it is transformed from its reduced form to its oxidized form. When an acceptor gains an electron, it changes from its oxidized form to its reduced form. Together, the oxidized and reduced forms of a redox component, such as lipoic acid and DHLA or CoQ-10 (ubiquinone) and reduced CoQ-10 (ubiquinol) are called “redox couples.”

Dihydrolipoic acid is the reduced (has electrons added) form of lipoic acid (thioctic acid). When DHLA is oxidized (has electrons removed) lipoic acid is produced. It should be understood that DHLA can be either the R or S enantiomer or it can be racemic. Likewise, lipoic acid can also be enantiomerically pure or racemic.

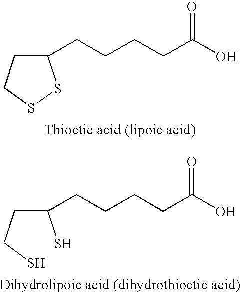

Likewise, ubiquinol is the reduced (has electrons added) form of ubiquinone (CoQ-10 for example). When ubiquinol is oxidized (has electrons removed), ubiquinone is produced.

Solutions of coenzyme Q-10 (CoQ-10) and reduced CoQ-10 with dihydrolipoic acid (DHLA) provide or maintain the reduced CoQ form. Interestingly, when about a molar amount of DHLA is combined with a molar equivalent of CoQ-10, the oxidized form of CoQ-10 is reduced to the reduced form of CoQ-10 (ubiquinol).

In the presence of about a molar equivalent of DHLA, generally, greater than 90% of the oxidized form of CoQ-10 is converted to the reduced form of CoQ-10 and in particular greater than 95%, more particularly, 96%, still more particularly, 97%, more particularly 98%, still more particularly, 99% conversion occurs, to a point where essentially no oxidized CoQ-10 remains. Excess DHLA can then serve as a solvent carrier and helps to stabilize the reduced CoQ-10, making shelf stable for extended periods of time.

In one aspect, the present invention provides a reduced coenzyme Q-10 (CoQ-10) composition that includes a sufficient amount of dihydrolipoic acid (DHLA) to reduce CoQ-10 to a reduced form of CoQ-10 in greater than 95% by weight in combination with garlic oil or one or more of the constituents of garlic oil.

The compositions according to the present invention can be present in liquid form. Otherwise, the composition can, at room temperature, be present as a gel or as a solid, dependent on the coenzyme Q concentration, but may become liquid at body temperature (37° C.).

The phrase “sulfide containing material” is intended to include sulfides, disulfides, trisulfides, tetrasulfides and at least pentasulfides.

The phrase “a sufficient amount of a sulfide containing material” is intended to mean the amount of a sulfide containing material needed to dissolve a known amount of a coenzyme Q material.

The mixture of the sulfide containing material and coenzyme Q material is generally a liquid. However, it is possible that concentrated compositions of the sulfide containing material and coenzyme Q material may result in a waxy or paste like material that will readily become liquid at body temperature as noted above.

Exemplary sulfide containing materials include, but are not limited to, sulfides, disulfides, trisulfides, tetrasulfides, or pentasulfides such as diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methylallylsulfide, dimethyltrisulfide, dimethyldisulfide, propylallyldisulfide, allylpropyltrisulfide, methylallyltetrasulfide, methyl allylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

In one aspect, the sulfide containing material is garlic oil. Garlic oil contains many of the above-identified sulfides. The garlic oil can be purified or non-purified. Garlic oil has been purified by distillation techniques and column chromatography.

It has been found that three components of garlic oil are especially useful for dissolving coenzyme Q materials. These include diallylsulfide, diallyltridsulfide and/or diallyldisulfide. Again, the components of the garlic oil can be purified or not prior to use.

The exemplary sulfide containing materials can be purchased having a purity of 95% or greater or can be purified by various methods, such as distillation or chromatography. In certain instances, it is advantageous to utilize purified sulfide containing material. Purity of the material generally is from about 90%, 91%, 92%, 93%, 94%, 95%, 96%, 97%, 98%, 99%, in particular, 99.5%, more particularly 99.9% or greater.

Interestingly, there appears to be a correlation that lower the molecular weight of the sulfide containing material, the greater degree of solubility of the coenzyme Q in the material.

Surprisingly it has been found that the combination of a coenzyme Q material(s) and garlic oil (or one or more of its components) can have an advantageous effect on the diminishment or prevention of the oxidation of LDL and thus conditions associated with the oxidation of LDL. The combination appears to be synergistic as evidenced by the data provided below.

In general the ratio of garlic oil (or one or more of components thereof) to a coenzyme Q material (or materials) is from about 10 milligrams (mg) to about 400 mg coenzyme Q material to 1 milliliter (ml) of garlic oil, or one or more of the components of garlic oil, more particularly from about 100 mg to about 300 mg and even more particularly from about 100 mg to about 150 mg per 1 ml of garlic oil or a component thereof.

The term “LDL” refers to low density lipoprotein and is well recognized in the art. Low-density lipoprotein (LDL) is a lipoprotein that transports cholesterol and triglycerides from the liver to peripheral tissues. LDL is one of the five major groups of lipoproteins; these groups include chylomicrons, very low-density lipoprotein (VLDL), intermediate-density lipoprotein (IDL), low-density lipoprotein, and high-density lipoprotein (HDL). Like all lipoproteins, LDL enables fats and cholesterol to move within the water based solution of the blood stream. LDL also regulates cholesterol synthesis at these sites. It commonly appears in the medical setting as part of a cholesterol blood test, and since high levels of LDL cholesterol can signal medical problems like cardiovascular disease, it is sometimes referred to as “bad cholesterol”.

LDL oxidation is related to diabetic complications, atherosclerosis, and other cardiovascular diseases.

Therefore, the present invention provides compositions and methods to treat or prevent diabetic and/or cardiovascular diseases. The invention also provides methods to manufacture a medicament to treat, reduce, or prevent a disease or condition described herein comprising the step of providing (such as administering) to a subject in need thereof, an effective amount (a therapeutically effective amount) of a coenzyme Q material and garlic oil (or one or more of the constituents of garlic oil), such that the disease or condition is treated, prevented, or reduced.

Administration of an effective amount of the combination of garlic oil (or one or more of the constituents thereof) and a coenzyme Q therefore provides a preventative or therapeutic method for one or more of the conditions noted throughout the specification.

An effective amount refers to that amount found to treat or prevent a physiological condition. This can be noted by the diminishment or prevention of one or more symptoms associated with the condition.

The compositions of the invention include a “therapeutically effective amount” or a “prophylactically effective amount” of one or more of the combination of sulfide(s) and coenzyme Q materials of the invention. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the sulfide containing material and coenzyme Q combination may vary according to factors such as the disease state, age, sex, and weight of the individual, and the ability of the therapeutic compound to elicit a desired response in the individual. A therapeutically effective amount is also one in which any toxic or detrimental effects of the therapeutic agent are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the combination and the particular therapeutic or prophylactic effect to be achieved, and (b) the limitations inherent in the art of compounding such an active combination for the treatment of sensitivity in individuals.

An exemplary, non-limiting range for a therapeutically or prophylactically effective amount of a sulfide containing/coenzyme Q composition of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

When the compounds of the present invention are administered as pharmaceuticals, to humans and mammals, they can be given per se or as a pharmaceutical composition containing, for example, 0.1 to 99.5% (more preferably, 0.5 to 90%) of the active combination, i.e., at least one sulfide containing compound and at least one coenzyme Q material, in combination with a pharmaceutically acceptable carrier.

The term “diabetic complications” is known in the art and refers to diabetes and its related symptoms such as cardiovascular diseases.

The term “atherosclerosis” is known in the art and refers to a disease affecting arterial blood vessels. It is a chronic inflammatory response in the walls of arteries, in large part due to the accumulation of macrophage white blood cells and promoted by low density lipoproteins (LDL) without adequate removal of fats and cholesterol from the macrophages by functional high density lipoproteins (HDL). It is commonly referred to as a “hardening” or “furring” of the arteries. It is caused by the formation of multiple plaques within the arteries.

Arteriosclerosis is a general term describing any hardening (and loss of elasticity) of medium or large arteries; arteriolosclerosis is any hardening (and loss of elasticity) of arterioles (small arteries); atherosclerosis is a hardening of an artery specifically due to an atheromatous plaque. Therefore, atherosclerosis is a form of arteriosclerosis.

The term “cardiovasular diseases” is known in the art and refers to arteriosclerosis, coronary artery disease, heart valve disease, arrhythmia, heart failure, hypertension, orthostatic hypotension, shock, endocarditis, diseases of the aorta and its branches, disorders of the peripheral vascular system, and congenital heart disease.

Additionally, carriers may be included with the sulfide containing material with the coenzyme Q material. Suitable carriers include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil; mustard seed oil, olive oil, sesame oil, soybean oil, corn oil, peanut oil, cottonseed oil, rice bran oil, babassu nut oil, palm oil, low erucic rapeseed oil, palm kernel oil, lupine oil, coconut oil, flaxseed oil, evening primrose oil, jojoba, tallow, beef tallow, butter, chicken fat, lard, dairy butterfat, shea butter or combinations thereof.

Non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carriers, beeswax can be used as a suitable carrier, as well as suspending agents such as silica (silicon dioxide).

Non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carriers, beeswax can be used as a suitable carrier, as well as suspending agents such as silica (silicon dioxide).

Additionally, limonene singly, and/or with other cyclic monoterpene containing essential oil(s), such as orange oil (which may contain 95% or more d-limonene) can be included with one or more carriers. Non-limiting examples of d-limonene containing oils include Lavindin, Peppermint, Ginger, Camphor, Geranium, Orange, Lemon, Lavender, Tea Tree, and Rosemary.

The formulations of the invention are considered dietary supplements useful to the increase the amounts of one or more sulfide containing materials, a coenzyme Q material and/or additional antioxidants in individuals in need thereof.

The formulations of the invention can also be used in cosmetic products.

Alternatively, the formulations of the invention are also considered to be nutraceuticals. The term “nutraceutical” is recognized in the art and is intended to describe chemical compounds found in foods that may prevent disease. For example, reduced CoQ-10 and antioxidants are such compounds.

The formulations of the invention can further include various ingredients to help stabilize, or help promote the bioavailability of the coenzyme Q material and/or amino acid(s), or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s), if present, are present in the composition of the invention in an amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s), if present, are present in the composition of the invention in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

Various additives can be incorporated into the present compositions. Optional additives of the present composition include, without limitation, phospholipids, L-carnitine, starches, sugars, fats, antioxidants, amino acids, proteins, flavorings, coloring agents, hydrolyzed starch(es) and derivatives thereof or combinations thereof.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, and green tea extract.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC).

Any dosage form, and combinations thereof, are contemplated by the present invention. Examples of such dosage forms include, without limitation, chewable tablets, elixirs, liquids, solutions, suspensions, emulsions, capsules, soft gelatin capsules, hard gelatin capsules, caplets, lozenges, chewable lozenges, suppositories, creams, topicals, ingestibles, injectables, infusions, health bars, confections, animal feeds, cereals, cereal coatings, and combinations thereof. The preparation of the above dosage forms are well known to persons of ordinary skill in the art.

For example, health bars can be prepared, without limitation, by mixing the formulation plus excipients (e.g., binders, fillers, flavors, colors, etc.) to a plastic mass consistency. The mass is then either extended or molded to form “candy bar” shapes that are then dried or allowed to solidify to form the final product.

Soft gel or soft gelatin capsules can be prepared, for example, without limitation, by dispersing the formulation in an appropriate vehicle (e.g. rice bran oil, DHLA and/or beeswax) to form a high viscosity mixture. This mixture is then encapsulated with a gelatin based film using technology and machinery known to those in the soft gel industry. The industrial units so formed are then dried to constant weight. Typically, the weight of the capsule is from about 100 to about 2500 milligrams and in particular weigh from about 1500 and about 1900 milligrams, and more specifically can weigh from about 1500 and about 2000 milligrams.

For example, when preparing soft gelatin shells, the shell can include from about 20 to 70 weight percent gelatin, generally a plasticizer and about 5 to about 60% by weight sorbitol. The filling of the soft gelatin capsule is liquid (principally limonene, in combination with rice bran oil and/or beeswax if desired) and can include, apart form the antioxidant actives, a hydrophilic matrix. The hydrophilic matrix, if present, is a polyethylene glycol having an average molecular weight of from about 200 to 2000. Further ingredients are optionally thickening agents. In one embodiment, the hydrophilic matrix includes polyethylene glycol having an average molecular weight of from about 200 to 2000, 5 to 15% glycerol, and 5 to 15% by weight of water. The polyethylene glycol can also be mixed with propylene glycol and/or propylene carbonate.

In another embodiment, the soft gel capsule is prepared from gelatin, glycerine, water and various additives. Typically, the percentage (by weight) of the gelatin is from about 30 and about 50 weight percent, in particular from about 35 and about 45 weight percent and more specifically about 42 weight percent. The formulation includes from about 15 and about 25 weight percent glycerine, more particularly from about 17 and about 23 weight percent and more specifically about 20 weight percent glycerine.

The remaining portion of the capsule is typically water. The amount varies from about 25 weight percent and about 40 weight percent, more particularly from about 30 and about 35 weight percent, and more specifically about 35 weight percent. The remainder of the capsule can vary, generally, from about 2 and about 10 weight percent composed of a flavoring agent(s), sugar, coloring agent(s), etc. or combination thereof. After the capsule is processed, the water content of the final capsule is often from about 5 and about 10 weight percent, more particularly 7 and about 12 weight percent, and more specifically from about 9 and about 10 weight percent.

As for the manufacturing, it is contemplated that standard soft shell gelatin capsule manufacturing techniques can be used to prepare the soft-shell product. Examples of useful manufacturing techniques are the plate process, the rotary die process pioneered by R. P. Scherer, the process using the Norton capsule machine, and the Accogel machine and process developed by Lederle. Each of these processes are mature technologies and are all widely available to any one wishing to prepare soft gelatin capsules.

Typically, when a soft gel capsule is prepared, the total weight is from about 250 milligrams and about 2.5 gram in weight, e.g., 400-750 milligrams. Therefore, the total weight of additives, such as vitamins and antioxidants, is from about 80 milligrams and about 2000 milligrams, alternatively, from about 100 milligrams and about 1500 milligrams, and in particular from about 120 milligrams and about 1200 milligrams. In particular, the soft gel capsule typically weighs from about 1000 milligrams and 1300 milligrams, wherein the percentage fill is about 50% of the entire weight of the capsule, i.e., from about 500 to about 650 milligrams fill weight. The fill weight includes the active ingredient(s), solubilizing agents, etc.

Preparation of the Soft Gel Capsules was Accomplished by Methods well known in the art including, but not limited to those described throughout the specification and in U.S. Pat. Nos. 6,616,942, 6,623,734 and pending U.S. Ser. Nos. 10/035,753 and 09/825,920, the contents of which are incorporated herein by reference in their entirety.

For example, a soft gel capsule can be prepared by mixing a garlic oil and a coenzyme Q material to provide a syrupy mixture. The mixture is then encapsulated within a gelatin capsule as described above.

Tablets, capsules, powders and/or solutions can include one or more of excipients, disintegrants, lubricants, binders, colorants, aggregation inhibitors, absorption enhancers, solubilizing agents, stabilizer and the like.

Excipients include, for example, white sugar, lactose, glucose, corn starch, mannitol, crystalline cellulose, calcium phosphate, calcium sulfate and the like.

Disintegrants include, for example, starch, agar, calcium citrate, calcium carbonate, sodium hydrogen carbonate, dextrin, crystalline cellulose, carboxymethylcellulose, tragacanth and the like.

Lubricants include, for example, talc, magnesium stearate, polyethylene glycol, silica, hardened vegetable oils and the like.

Binders include, for example, ethylcellulose, methylcellulose, hydroxypropylmethylcellulose, tragacanth, shellac, gelatin, gum arabic, polyvinylpyrrolidone, polyvinyl alcohol, polyacrylic acid, polymethacrylic acid, sorbitol and the like.

The present invention also provides packaged formulations of a coenzyme Q material, such as reduced CoQ-10 and/or CoQ-10, and a sulfide containing compound and instructions for use of the tablet, capsule, elixir, etc. Typically, the packaged formulation, in whatever form, is administered to an individual in need thereof that requires an increase in the amount of a coenzyme Q material and/or a sulfide containing material in the individual's diet. Typically, the dosage requirement is from about 1 to about 4 dosages a day.

CoQ-10 has been implicated in various biochemical pathways and is suitable for the treatment of cardiovascular conditions, such as those associated with, for example, statin drugs that effect the body's ability to product CoQ-10 naturally. CoQ-10 has also been implicated in various periodontal diseases. Furthermore, CoQ-10 has been implicated in mitochondrial related diseases and disorders, such as the inability to product acetyl coenzyme A, neurological disorders, for example, such as Parkinson's disease and, Prater-Willey syndrome, migraine headaches and headaches.

The following paragraphs enumerated consequently from 1 through 48 provide for various aspects of the present invention. In one embodiment, in a first paragraph (1), the present invention provides a composition comprising a coenzyme Q, or a reduced coenzyme Q or mixtures thereof and a sufficient amount of a sulfide containing material suitable to dissolve the coenzyme Q, or reduced coenzyme Q or mixtures thereof.

2. The composition of claim 1, wherein from about 20 percent and about 70 percent coenzyme Q is dissolved in the sulfide containing material on a weight basis.

3. The composition of either of claims 1 or 2, wherein the sulfide containing material is garlic oil.

4. The composition of any of claims 1 through 3, wherein the garlic oil is a purified garlic oil.

5. The composition of any of claims 1 through 4, wherein the sulfide containing material is a sulfide, a disulfide, a trisulfide, a tetrasulfide, a pentasulfide or mixtures thereof.

6. The composition of claim 5, wherein the sulfide, disulfide, trisulfide, tetrasulfide, or pentasulfide containing material is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methylallyl sulfide, dimethyltrisulfide, dimethyldi sulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

7. The composition of claim 6, wherein the sulfide containing material is a purified material.

8. The composition of any of claims 1 through 7, wherein the coenzyme Q or reduced coenzyme Q has the formula

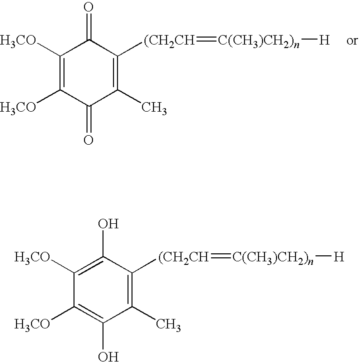

wherein n is from 1 to about 12.

9. The composition of claim 8, wherein n is 10.

10. The composition of any of claims 1 through 9, further including an antioxidant.

11. The composition of claim 10, wherein the antioxidant is dihydrolipoic acid.

12. The composition of any of claims 1 through 11, wherein the composition is encapsulated within a soft gelatin capsule.

13. A method to increase the bioavailability of a coenzyme Q, or a reduced coenzyme Q, or mixtures thereof, comprising the step of combining a sufficient amount of a sulfide containing material suitable to dissolve the coenzyme Q, or reduced coenzyme Q or mixtures thereof.

14. The method of claim 13, wherein from about 20 percent and about 70 percent coenzyme Q is dissolved in the sulfide containing material on a weight basis.

15. The method of either of claims 13 or 14, wherein the sulfide containing material is garlic oil.

16. The method of any of claims 13 through 15, wherein the garlic oil is a purified garlic oil.

17. The method of any of claims 13 through 16, wherein the sulfide containing material is a sulfide, a disulfide, a trisulfide, a tetrasulfide, a pentasulfide or mixtures thereof.

18. The method of claim 17, wherein the sulfide, disulfide, trisulfide, tetrasulfide, or pentasulfide containing material is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methyl allyl sulfide, dimethyltrisulfide, dimethyl disulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

19. The method of claim 18, wherein the sulfide containing material is a purified material.

20. The method of any of claims 13 through 19, wherein the coenzyme Q or reduced coenzyme Q has the formula

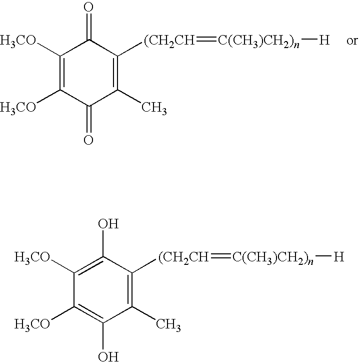

wherein n is from 1 to about 12.

21. The method of claim 20, wherein n is 10.

22. The method of any of claims 13 through 21, further including an antioxidant.

23. The method of claim 22, wherein the antioxidant is dihydrolipoic acid.

24. The method of any of claims 13 through 23, wherein the bioavailability of the coenzyme Q, reduced coenzyme Q or mixture thereof is increased by about 15 percent to about 1500 percent relative to a composition that does not include a sulfide containing material.

A method to treat mitochondrial related diseases and disorders, Parkinson's disease, Prater-Willey syndrome, cardiovascular disease, congestive heart failure, migraine headaches or headaches comprising the step of administering to a subject in need thereof, an effective amount of a coenzyme Q, or a reduced coenzyme Q, or mixtures thereof and a sufficient amount of a sulfide containing material suitable to dissolve the coenzyme Q, or reduced coenzyme Q or mixtures thereof, such that the effective amount of the coenzyme Q, or the reduced coenzyme Q or mixtures thereof are delivered to treat mitochondrial related diseases and disorders, Parkinson's disease, Prater-Willey syndrome, cardiovascular disease, congestive heart failure, migraine headaches or headaches.

26. The method of paragraph 25, wherein from about 20 percent and about 70 percent coenzyme Q is dissolved in the sulfide containing material on a weight basis.

27. The method of either of paragraphs 25 or 26, wherein the sulfide containing material is garlic oil.

28. The method of any of paragraphs 25 through 27, wherein the garlic oil is a purified garlic oil.

29. The method of any of paragraphs 25 through 28, wherein the sulfide containing material is a sulfide, a disulfide, a trisulfide, a tetrasulfide, a pentasulfide or mixtures thereof.

30. The method of paragraph 29, wherein the sulfide, disulfide, trisulfide, tetrasulfide, or pentasulfide containing material is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methyl allyl sulfide, dimethyltrisulfide, dimethyldi sulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

31. The method of paragraph 30, wherein the sulfide containing material is a purified material.

32. The method of any of paragraphs 25 through 31, wherein the coenzyme Q or reduced coenzyme Q has the formula

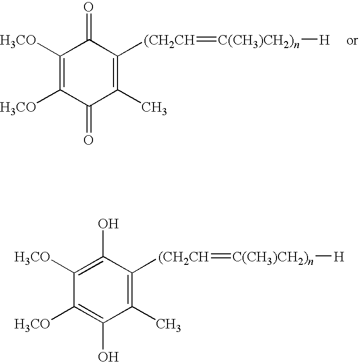

wherein n is from 1 to about 12.

33. The method of paragraph 32, wherein n is 10.

34. The method of any of paragraphs 25 through 33, further including an antioxidant.

35. The method of paragraph 34, wherein the antioxidant is dihydrolipoic acid.

36. The method of any of paragraphs 25 through 35, wherein the bioavailability of the coenzyme Q, reduced coenzyme Q or mixture thereof is increased by about 15 percent to about 1500 percent relative to a composition that does not include a sulfide containing material.

37. A method to treat or prevent oxidation of LDL in a subject, comprising providing to a subject in need thereof, a sufficient amount of a sulfide, disulfide, trisulfide, tetrasulfide, or pentasulfide containing material and a coenzyme Q, such that oxidation of LDL in the subject is treated or prevented.

38. The method of paragraph 37, wherein the sulfide is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methylallylsulfide, dimethyltrisulfide, dimethyldisulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

39. The method of either of paragraphs 37 or 38, wherein the sulfide, disulfide, trisulfide, tetrasulfide, or pentasulfide containing material is garlic oil.

40. A method to treat or prevent a cardiovascular disease in a subject, comprising providing to a subject in need thereof, a sufficient amount of a sulfide, disulfide, trisulfide, tetrasulfide, or pentasulfide containing material and a coenzyme Q, such that the cardiovascular disease in the subject is treated or prevented.

41. The method of paragraph 40, wherein the sulfide is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methylallylsulfide, dimethyltrisulfide, dimethyldisulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

42. The method of either of paragraphs 40 or 41, wherein the sulfide, disulfide, trisulfide, tetrasulfide, or pentasulfide containing material is garlic oil.

43. A method to treat or prevent diabetes in a subject, comprising providing to a subject in need thereof, a sufficient amount of a sulfide, disulfide, trisulfide, tetrasulfide, or pentasulfide containing material and a coenzyme Q, such that diabetes in the subject is treated or prevented.

44. The method of paragraph 43, wherein the sulfide is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methylallylsulfide, dimethyltrisulfide, dimethyldisulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

45. The method of either of paragraphs 43 or 44, wherein the sulfide, disulfide, trisulfide, tetrasulfide, or pentasulfide containing material is garlic oil.

46. A method to treat or prevent atherosclerosis in a subject, comprising providing to a subject in need thereof, a sufficient amount of a sulfide, disulfide, trisulfide, tetrasulfide, or pentasulfide containing material and a coenzyme Q, such that atherosclerosis in the subject is treated or prevented.

47. The method of paragraph 46, wherein the sulfide is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methylallylsulfide, dimethyltrisulfide, dimethyldisulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

48. The method of either of paragraphs 46 or 47, wherein the sulfide, disulfide, trisulfide, tetrasulfide, or pentasulfide containing material is garlic oil.

The following examples are intended to be illustrative only and should not be considered limiting.

### Methods for Absorption Testing Using Caco-2 Cells

1. Cell Culture

1.1. Maintenance of Caco-2 Cells

Caco-2 HTB-37 (human colon adenocarinoma), obtained from ATCC, were cultured in DMEM (Dulbecco/Vogt modified Eagle's minimal essential medium) containing 20% FCS (Fetal Calf Serum), 1% nonessential amino acids, 0.83 mM L-glutamine and 1% penicillin-streptomycin at 37° C. in a humified atmosphere of CO₂. Cells were grown in 75 cm² culture-flasks (T75) and their medium was replaced every third day. As soon as confluency was reached—once a week—they were split using trypsin.

1.2. Subculturing Caco-2 Cells

After discarding the old medium, the monolayer was washed with 10 mL PBS. Subsequently, 3 mL trypsin was added in order to detach the cells. As Caco-2 cells have shown to grow at a slow pace, it is reasonable to split them 1:4 by mixing them vigorously with 9 mL DMEM and leaving 3 mL cell-suspension in the flask which was refilled with 17 mL DMEM.

The remaining 9 mL cell-suspension was used for experiments and was therefore transferred into a 15 mL falcon. After the cells had been counted by using a Neubauer-chamber, cells were seeded into 6 Well Thin Certs and 6 Wells at a density of 3×10⁵ and 6.76×10⁵, respectively. DMEM was added so that the final volume in 6 Wells reached 5 mL and 3 mL in 6 Well Thin Certs. Cultures were used for experiments approximately 14 days post confluency because the marker enzymes, alkaline phosphatase and sucrase show maximum differentiation at this time.

2. Experimental Design

2.1. Preparation of Emulsion

CoQ10 was dissolved/suspended in safflower oil, dissolved in garlic oil or in crystalline form, was transferred into plastic tubes and 0.4 g olive oil was added. The sample was vortexed and sonicated for 10 min. Subsequently the sample was left in a water-bath at 80° C. for 10 min. 100 mL 10 mM bile salts in 150 mM NaCl-solution were added. (A 10 mM bile salt-solution corresponds to the average bile salt concentration in the duodenum during digestion.)

The optimum amount of the emulsifier lecithin had to be determined individually for each sample. A molar ratio of lecithin: bile salts=0.4 turned out to be appropriate in most cases (0.4236 g lecithin was added to 100 mL 10 mM bile salt-solution). Afterwards the sample was shaken vigorously and sonicated at 40° C. Continuous shaking was a prerequisite for adequate dissolution.

2.2. Preparation of Suspension

For CoQ10 in crystalline form also a simple suspension in a bile salt/NaCl solution was prepared and introduced into the absorption test with no further treatment.

2.3. Samples Prepared

| | | | | Final conc. |
|---|---|---|---|---|
| Compound | Solvent | Treatment | Further Treatment | (mg/100 mL) |
| CoQ10 | — | suspension | Dilution DMEM | 25 |
| CoQ10 | — | emulsion | Dilution DMEM | 25 |
| CoQ10 | safflower oil | emulsion | Dilution DMEM | 25 |
| CoQ10 | garlic oil | emulsion | Dilution DMEM | 25 |
| (30%) | | | | |
| CoQ10 | garlic oil | emulsion | Dilution DMEM | 25 |
| (40%) | | | | |

The absorption of CoQ10 was tested with various preparations.

CoQ10 was also made available in its reduced state (ubiquinol) by addition of Dihydrolipoic Acid (DHLA) or Ascorbyl-palmitate (AP) into the garlic oil solution of CoQ₁₀.

| | | | | Final conc. |
|---|---|---|---|---|
| Compound | Solvent | Treatment | Further Treatment | (mg/100 mL) |
| CoQ10 (30%) | garlic oil | emulsion | Addition od | 25 (DHLA) |
| | | | DHLA. Dilution | 5 (CoQ10) |
| | | | DMEM | |
| CoQ10 (30%) | garlic oil | emulsion | Addition of AP. | 25 (AP) |
| | | | Dilution DMEM | 5 (CoQ10) |

2.4. Cellular Uptake

Coenzyme Q10-micellar solutions were diluted 1:3 (v/v) in DMEM. Before the addition of 1-1.5 mL test solution, mono-layers of the cells were washed with 2 mL PBS. Cell cultures were incubated at 37° C. for up to 2 h.

After 30, 60 and 120 minutes, plates (3 plates/time point) were put on ice and the media were collected separately. Wells were washed with 1 mL PBS several times. Then, cells were harvested in 1-2 mL PBS. Cells were then centrifuged at 1,000 g for 6 min at 4° C. The supernatant was discarded. The cells were broken by sonication in the presence of 1 mL 2-propanol/hexane (1/9, v/v). The organic layer containing CoQ10 (oxidized and reduced) was submitted to HPLC-analysis.

2.5. HPLC-Analysis

A reversed-phase column (Spherimage-80) with 5 μm pore size and 4.6 mm in length was used. The elution was isocratic with a solvent containing: 6.8 g sodium acetate, 15 mL glacial acetic acid, 15 mL 2-propanol, 695 mL methanol, 275 mL hexane at a flow rate of 1 mL/min. Coenzyme Q10 (oxidized and reduced) were monitored at 275 nm.

2.6. Data Evaluation

CoQ10 was determined in the media (outside the cells) and in the cells. Both, oxidized and reduced CoQ10, were quantified.

As the content of CoQ10 is subject to variation depending on the sample preparation the uptake into cells is expressed as relative difference between inside the cells and outside the cells (%-uptake). For all samples in a series the media concentration of CoQ10 at time 0 was taken as reference value. Analysis showed that the content of CoQ10 in the media was stable during the incubation conditions.

The %-uptake at each time point was then subjected to non-compartmental pharmacokinetic analysis (Cₘₐₓ, AUC₀₋₁₂₀ₘᵢₙ).

Results

1.1. Test Results for the Comparison of Various CoQ10 Preparations

The results presented below represent the sum of CoQ10 in reduced and oxidized form.

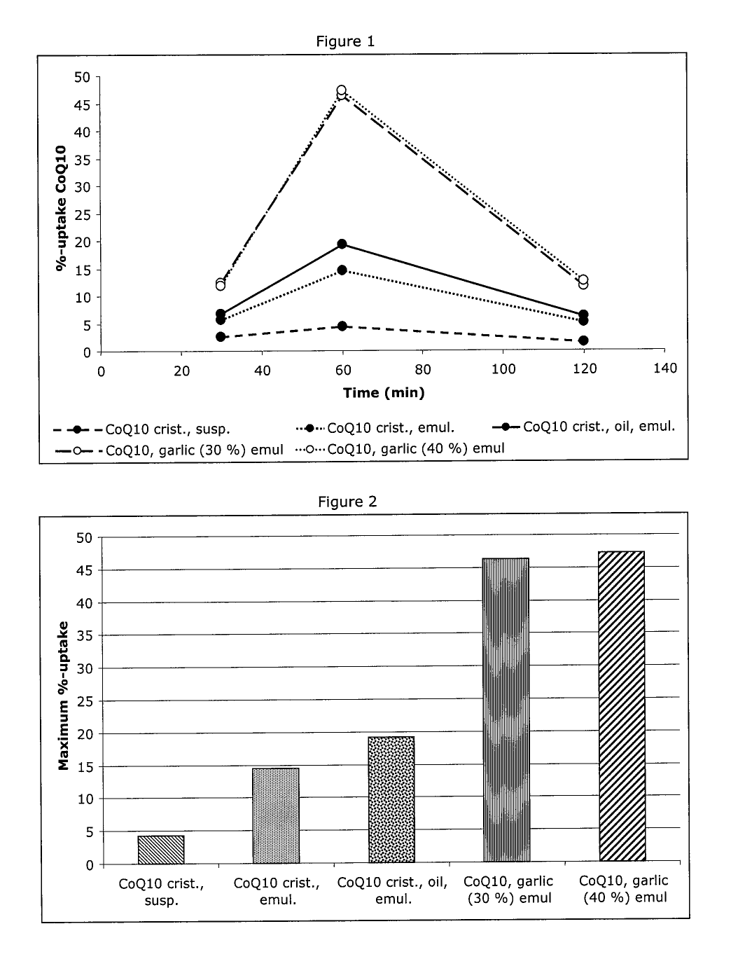

TABLE 1
%-uptake of CoQ10 from various sample preparations (See also FIG. 1)

| | Time (min) | | |
|---|---|---|---|
| Sample Description | 30 | 60 | 120 |
| CoQ10 crist., susp. | 2.6 | 4.3 | 1.5 |
| CoQ10 crist., emul. | 5.7 | 14.5 | 5.1 |
| CoQ10 crist., oil, emul. | 6.8 | 19.2 | 6.2 |
| CoQ10, garlic (30%) emul. | 12.4 | 46.3 | 11.7 |
| CoQ10, garlic (40%) emul. | 11.9 | 47.2 | 12.5 |

CoQ10 crist. Susp. = CoQ10 crystals suspended in aqueous medium
CoQ10 crist., emul. = CoQ10 crystals in a stable emulsion
CoQ10 crist., oil, emul. = CoQ10 crystals, dissolved in fat and then emulsified
CoQ10, garlic (30%) emul. = CoQ10 dissolved in garlic oil (essential oil) and then emulsified.

TABLE 2
Pharmacokinetic parameters for CoQ10 from various sample
preparations (See also FIG. 2)

| | Cmax | AUC0-120 |
|---|---|---|
| Sample Description | (%-uptake) | (%-uptake × min.) |
| CoQ10 crist., susp. | 4.3 | 278 |
| CoQ10 crist., emul. | 14.5 | 891 |
| CoQ10 crist., oil, emul. | 19.2 | 1152 |
| CoQ10, garlic (30%) emul. | 46.3 | 2621 |
| CoQ10, garlic (40%) emul. | 47.2 | 2678 |

As seen, dissolution of CoQ10 in garlic oil yields substantially higher absorption values when compared to CoQ10 dissolved/suspended in safflower oil and to CoQ10 subjected in crystalline form as suspension.

1.2. Comparison of Absorption of Reduced and Oxidized CoQ10

The dissolution of CoQ10 in garlic oil (30%) was further tested after addition of either DHLA or Ascorbyl-palmitate (AP) in a 5-fold excess to CoQ10 (on a molar basis).

The solutions were left for up to 4 days at room temperature, with continuous stirring and protected from light to provide time for the reaction. After addition of AP, complete reduction (>90% reduced CoQ10) was observed after 1.5 days. After addition of DHLA, complete reduction was observed after 4 days.

After complete reduction, the incubations were performed as described above. The data analysis consists of the sum of reduced and oxidized CoQ10 found in the samples.

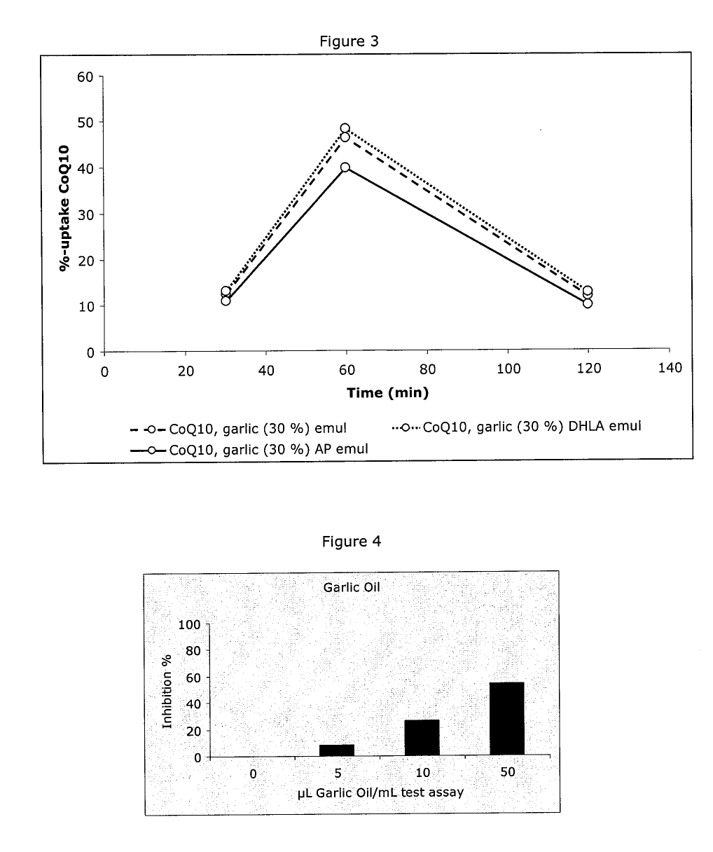

Table 3 and FIG. 3 provide the %-uptake of CoQ10 into CaCo2-cells.

TABLE 3
%-uptake of CoQ10 from reduced sample preparations

| | Time (min) | | |
|---|---|---|---|
| Sample Description | 30 | 60 | 120 |
| CoQ10, garlic (30%) emul | 12.4 | 46.3 | 11.7 |
| CoQ10, garlic (30%) DHLA, emul | 13.1 | 48.2 | 12.6 |
| CoQ10, garlic (30%) AP, emul | 10.8 | 39.7 | 9.86 |

TABLE 4
Pharmacokinetic parameters for CoQ10 from reduced sample
preparations

| | | AUC0-120 |
|---|---|---|
| | Cmax | (%-uptake × |
| Sample Description | (%-uptake) | min.) |
| CoQ10, garlic (30%) emul | 46.3 | 2621 |
| CoQ10, garlic (30%) DHLA, emul | 48.2 | 2744 |
| CoQ10, garlic (30%) AP, emul | 39.7 | 2244 |

Prevention of LDL Oxidation

Human LDL were obtained by ultracentrifugation from fresh human blood, followed by dialysis against in 10 mM PBS (pH 7.4) at 4° C. in the dark for 24 h. LDL (0.1 mg/ml) were mixed with different amounts of active constituents. Reaction was initiated by adding a solution of CuSO₄ (10 μM); samples were then incubated at 37° C. for 22 h. The formation of conjugated diene was measured at 234 nm using a Hewlett-Packard spectrophotometer (Agilent, Palo Alto).

Data evaluation was performed by comparison of the slope of increase in diene-formation. The steepest slope (i.e. without addition of anti-oxidants) was set to 100%. Results are given as relative %.

The lag-time until start of diene formation was estimated from the kinetic curves obtained. Results are given in minutes.

Part 1

LDL-Oxidation in Presence of Garlic Oil

Result 1

TABLE 5

| μL/mL of garlic oil | Residual activity/% | Inhibition/% |
|---|---|---|
| 0 | 100 | 0 |
| 5 | 92 | 8 |
| 10 | 74 | 26 |
| 50 | 46 | 54 |

As indicated by Table 5, garlic oil can inhibit LDL-oxidation by about 54% at a concentration of 50 μL/mL in test assay solution (Concentration of LDL was 0.1 mg/ml).

Part 2

LDL-Oxidation in Presence of Coenzyme Q10

TABLE 6

| mg/mL of CoQ10 | Residual activity/% | Inhibition/% |
|---|---|---|
| 0 | 100 | 0 |
| 0.1 | 87 | 13 |
| 1 | 57 | 43 |
| 10 | 32 | 68 |

As indicated by Table 6, CoQ-10 can inhibit LDL-oxidation (at an LDL concentration of 0.1 mg/ml) by about 43% at a concentration of 1 mg/mL in test assay solution.

Part 3

LDL-Oxidation in Presence of Garlic Oil and Coenzyme Q10

TABLE 7

| μL/mL garlic oil//mg/mL | | |
|---|---|---|
| CoQ10 | Residual activity/% | Inhibition/% |
| 0 | 100 | 0 |
| 10//0.1 | 54 | 46 |
| 10//1 | 32 | 68 |
| 10//10 | 10 | 90 |

As noted in Table 7 and FIG. 6, the combination garlic oil and coenzyme Q10 inhibits LDL-oxidation by up to 90% at a concentration of 10 μL garlic oil/mL and 10 mg coenzyme Q10/mL per 0.1 mg of LDL.

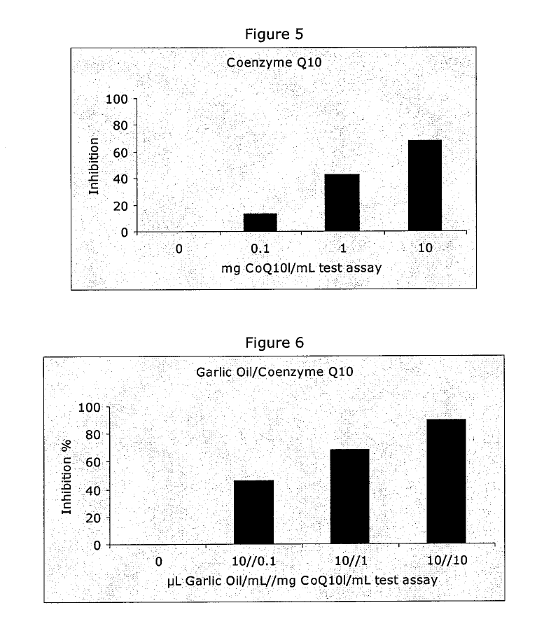

According to the data in Tables 5 through 7 and FIGS. 4 through 6, a synergistic effect of garlic oil and CoQ10 was found to inhibit the oxidation of LDL. For example, the combination of (10 μL garlic oil and 0.1 mg CoQ10)/mL provided 45% inhibition of oxidation of LDL (0.1 mg LDL) which is greater than the sum of 26% and 13% of inhibition of each antioxidant individually.

Result 2

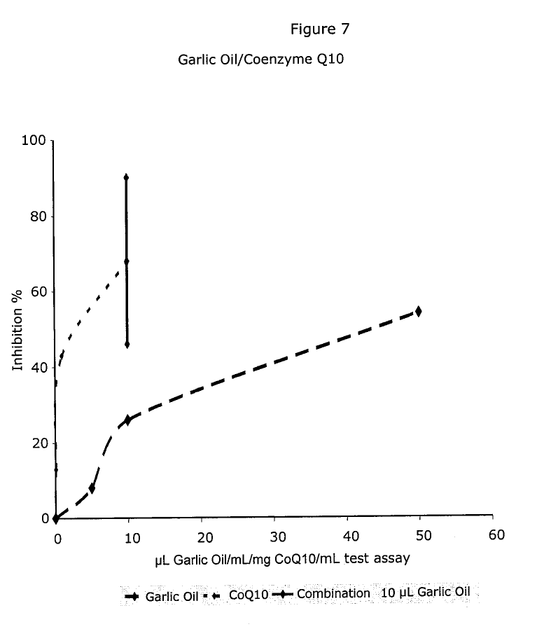

The following shows a relative equivalent relationship between garlic oil and CoQ10 by inhibiting LDL-oxidation in presence of garlic oil and/or coenzyme Q10. Based upon parts 1, 2 and 3 noted above, the data in FIG. 7 can be calculated.

Based on FIG. 7, 1 mg CoQ10 with garlic oil is equivalent to 10 mg CoQ10 without garlic oil when combined with 0.1 mg LDL per ml of solution.

Result 3

Determination of lag time provides the following results noted in Table 8. The data in Table 8 demonstrate a synergistic effect in comparison to the individual components versus the combination of garlic oil and Co Q10 as noted by the lag time to the start of LDL-oxidation (at a LDL concentration of 0.1 mg/ml).

TABLE 8

| | Lag Time (min) |
|---|---|
| Garlic Oil (10 μL/mL) | 121 |
| Co Q10 (1 mg/mL) | 146 |
| Combination (10 μL/mL//1 mg/mL) | 382 |

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

I claim:

1. A composition comprising a coenzyme Q, or a reduced coenzyme Q or mixtures thereof and a sufficient amount of a sulfide containing material suitable to dissolve the coenzyme Q, or reduced coenzyme Q or mixtures thereof.

2. The composition of claim 1, wherein from about 10 milligrams to about 400 milligrams coenzyme Q is dissolved in 1 milliliter of the sulfide containing material on a weight basis.

3. The composition of claim 1, wherein the sulfide containing material is garlic oil.

4. The composition of claim 3, wherein the garlic oil is a purified garlic oil.

5. The composition of claim 1, wherein the sulfide containing material is a sulfide, a disulfide, a trisulfide, a tetrasulfide, a pentasulfide or mixtures thereof.

6. The composition of claim 5, wherein the sulfide, disulfide, trisulfide, tetrasulfide, or pentasulfide containing material is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methyl allylsulfide, dimethyltrisulfide, dimethyldi sulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

7. A method to increase the bioavailability of a coenzyme Q, or a reduced coenzyme Q, or mixtures thereof, comprising the step of combining a sufficient amount of a sulfide containing material suitable to dissolve the coenzyme Q, or reduced coenzyme Q or mixtures thereof.

8. The method of claim 7, wherein the sulfide containing material is garlic oil.

9. The method of claim 7, wherein the sulfide containing material is a sulfide, a disulfide, a trisulfide, a tetrasulfide, a pentasulfide or mixtures thereof.

10. The method of claim 9, wherein the sulfide, disulfide, trisulfide, tetrasulfide, or pentasulfide containing material is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methyl allyl sulfide, dimethyltrisulfide, dimethyldi sulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

11. A method to treat or prevent a cardiovascular disease in a subject, comprising providing to a subject in need thereof, a sufficient amount of a sulfide, disulfide, tri sulfide, tetrasulfide, or pentasulfide containing material and a coenzyme Q, such that the cardiovascular disease in the subject is treated or prevented.

12. The method of claim 11, wherein the sulfide is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methylallylsulfide, dimethyltrisulfide, dimethyldisulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

13. The method of claim 11, wherein the sulfide, disulfide, trisulfide, tetrasulfide, or pentasulfide containing material comprises garlic oil.

14. A method to treat or prevent diabetes in a subject, comprising providing to a subject in need thereof, a sufficient amount of a sulfide, disulfide, trisulfide, tetrasulfide, or pentasulfide containing material and a coenzyme Q, such that diabetes in the subject is treated or prevented.

15. The method of claim 14, wherein the sulfide is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methylallylsulfide, dimethyltrisulfide, dimethyldisulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

16. The method of claim 14, wherein the sulfide, disulfide, trisulfide, tetrasulfide, or pentasulfide containing material comprises garlic oil.

17. A method to treat or prevent atherosclerosis in a subject, comprising providing to a subject in need thereof, a sufficient amount of a sulfide, disulfide, trisulfide, tetrasulfide, or pentasulfide containing material and a coenzyme Q, such that atherosclerosis in the subject is treated or prevented.

18. The method of claim 17, wherein the sulfide is one of diallylsulfide, allylmethylsulfide, allyethylsulfide, diallyltrisulfide, methylallyldisulfide, ethylallyldisulfide, diallyldisulfide, methylallyltrisulfide, diallyltrisulfide, ethylallyltrisulfide, diallyltetrasulfide, ajoene, 2-vinyl-4H-1,3-dithiin, 3-vinyl-5H-1,2-dithiin, methylallylsulfide, dimethyltrisulfide, dimethyldisulfide, propylallyldisulfide, allylpropyltrisulfide, methylallyltetrasulfide, methylallylpentasulfide, 6-methyl-1-thia-2,4-cyclohexadiene, 3-methyl-1,2-dithia-3-cyclopentene, 4-methyl-1,2-dithia-3-cyclopentene, 4-vinyl-1,2,3-trithia-5-cyclohexene, 3-vinyl-1,2-dithia-4-cyclohexene, dipropenyldisulfide, dithio-(propenyl)-propionate, 2-ethyltetrahydrothiophene or mixtures thereof.

19. The method of claim 17, wherein the sulfide, disulfide, trisulfide, tetrasulfide, or pentasulfide containing material comprises garlic oil.
